(19) United States
(12) Patent Application Publication
(10) Pub. No.: US 2014/0236104 A1
(43) Pub. Date: Aug. 21, 2014

(54) PUNCH-REDUCING CANNULA

(71) Applicant: Hans Haindl, Wennigsen (DE)

(72) Inventor: Hans Haindl, Wennigsen (DE)

(21) Appl. No.: 14/241,601

(22) PCT Filed: Aug. 31, 2012
(86) PCT No.: PCT/EP2012/066945

§ 371 Date: Feb. 27, 2014

(30) Foreign Application Priority Data
Aug. 31, 2011 (DE) 10 2011 112 021.5

(51) Int. Cl. A61M 5/32

(52) U.S. Cl. CPC A61M 5/3286; A61M 2207/00
USPC 604/274; 264/162; 156/211

(57) ABSTRACT

The present invention relates to a new bevel or grinding for a punch-reducing cannula, i.e. a cannula which reduces punching particles, and, in particular, to a punch-reducing cannula having a beveled or ground end as well as to a method for manufacturing a cannula which reduces punching particles.

The present invention relates to a new bevel or grinding for a punch-reducing cannula, i.e. a cannula which reduces punching particles, and, in particular, to a punch-reducing cannula having a beveled or ground end as well as to a method for manufacturing a cannula which reduces punching particles.

Medical cannulas used for puncturing skin or other materials generally tend to separate punching particles from the skin or other materials. Usually, this is extraneous, for example when it comes to intravenous or intramuscular injections. Yet, there are various medical applications of cannulas where this process becomes a problem. Such applications include, e.g., the puncturing of joints (intraarticular puncturing) or spinal puncturing, in which cases the intrusion of never entirely germ-free skin particles can cause infections. Problems can also arise during puncturing of so-called implantable port catheters or pumps, where a silicone membrane is punctured under the skin, which is supposed to seal tightly after removal of the cannula. If punching particles are constantly separated during these punctures, early failure of the silicone membrane's capability to reseal will be the result. Moreover, punching particles inside these ports or pumps are also unfavorable, because they can, for instance, cause blockage.

There are numerous cannula bevels or grindings which allow reducing the punching angle. These bevels generally work up to a certain cannula diameter. The larger and the more thin-walled the cannula is, the more difficult it becomes to constructively prevent the punching of particles.

Documents U.S. Pat. No. 2,746,454, EP 0 301 246 A1, DE 42 26 476 C1, EP 0 443 630 A1 and WO 94/03223 describe, for example, cannulas having a rigid cannula tube which is beveled at the tip and which comprises two further facet grindings arranged angularly towards each other on the front half of the beveled end. The cannula tip of EP 0 301 246 is bent beyond the cannula's center axis and into the region between two imaginary parallel lines which extend the inner and outer surface of the cannula tube in a forward direction. The rear cutting edge of the basic grinding is rounded inwardly. The cannula tip according to DE 42 26 476 C1 is bent between the imaginary extensions of the two opposing inner surfaces of the cannula tube. Additionally, the entire basic grinding is rounded bluntly all the way up to the beginning of the facet grinding. These prior art cannulas were partially invented under the aspect that, when puncturing a blood vessel, such a bent cannula tip is less prone to puncture an adjacent vessel wall. Documents EP 0 755 690 A2 and DE 41 01 231 A1 describe other cannulas designed for the same purpose. Most of these prior art cannulas are also used for puncturing implantable port catheters due to the reduced punching effect.

The oldest cannula which reduces punching particles and which is generally referred to in this context is the cannula according to Huber (cf. U.S. Pat. No. 2,746,454), whose cannula tube is kinked above the bevel. The underlying idea is that the sharp distal end of the bevel is placed in the “shadow” of the cannula tip. Yet, this idea is false for the cannula's insertion direction is not determined by the user, but by the geometry of the bevel. Studies by Müller and Zierski (cf. Klinische Wochenschr. Vol. 66 (1988), pages 963-969) provide convincing evidence therefor.

Improved cannula bevels or grindings by Haindl (cf., e.g., EP 0 301 246 A1, DE 42 26 476 C1, EP 0 443 630 A1 and WO 94/03223) with a curvature inside the grinding which is complemented by blunting measures on parts of the grinding achieve significantly better results and, with a diameter of up to approximately 0.9 mm these cannulas do not punch out any particles.

Document WO 92/05816 A1 also discloses an allegedly punch-reducing cannula with particular emphasis on a fusion-rounded rear cutting edge. Admittedly, document WO 92/05816 A1 also discloses a bent cannula tip with a rear bevel, yet a comparison of the views provided in FIGS. 2A and 2B shows that the cannula is bent first and beveled afterwards, which, from a manufacturing point of view, is quite complex and can lead to detrimental results, because the desired bevel angles can be better adjusted with unbent cannulas.

The so-called pencil-point cannula having a lateral opening is one of various approaches to create a non-punching cannula. These cannulas have a conic tip like a pencil and thanks to the lateral arrangement of the opening no silicone particles can be planed off. Yet, the disadvantage of these cannulas is that they make puncturing painful, because they lack a sharp cutting edge. This deficiency can be remedied by grinding the conic tip of the pencil point cannula in a triangular shape resulting in a so-called trocar grinding (cf., e.g., EP 1 036 571 A2). The problem is, though, that the manufacturing of these cannulas is relatively complex and expensive, which is why these cannulas failed to prevail on a larger scale.

A further problem arises with respect to implantable port system and pumps. Here, the cannula tip typically strikes a more or less hard surface. The grounds or baffle plates with which the cannula comes into contact are either made of hard and resistant plastics, metal or ceramics. Most available grindings have very pointed cutting edges which often bend and form hooks when striking a hard surface. These hooks can damage the silicone membrane when the cannula is removed and, in some cases, be painful to the patient as they can get stuck in the patient's skin.

It is an object of the present invention to provide a punch-reducing cannula preferably without generating particles when penetrating an organic or artificial membrane substrate. It is a further object of the invention to provide a punch-reducing cannula, preferably a port cannula, with a tip that does not retain a hook-like deformation when striking a hard baffle plate. A further object of the invention is to provide a simple and cost-efficient method for manufacturing a punch-reducing cannula.

This/These object(s) is/are achieved by the features of the independent claims. Preferred embodiments of the invention are described in the dependent claims.

Accordingly, the invention relates to a cannula having a longitudinal cannula axis and a cannula tip with a beveled end which comprises two section grindings and a basic grinding defining a first grinding surface or ground section. The section grindings respectively define second and third ground sections which intersect at an intersection line (which forms a line segment having a defined length), wherein the intersection line is substantially perpendicular to the cannula's longitudinal axis. In the context of the present invention “substantially perpendicular” refers to the angular range of 45° to 135°. According to the present invention, the intersection line is, thus, substantially perpendicular to the longitudinal cannula axis when the vertical component of the intersection line (with respect to the plane through which the longitudinal cannula axis extends) is greater than the horizontal component of the intersection line. In other words, the intersection line is substantially perpendicular to the cannula's longitudinal axis when the projection of the intersection line onto the longitudinal cannula axis is smaller than or equal to the projection of the intersection line onto the plane which is perpendicular to the longitudinal cannula axis.

In other words, the cutting edge of the cannula according to the present invention is substantially perpendicular to the longitudinal cannula axis before bending. Hence, contrary to known cannulas the claimed cannula grinding or bevel does not end in a triangular tip, but in a sharp cutting edge which is substantially perpendicular to the cannula wall and approximately as broad as the cannula's wall thickness. From a technical perspective this means that the facet angle γ, as, for example, defined in the standards DIN 13097-4:2009-08 (D) and EN ISO 7864, is around 0°. Hence, the bevel of the present invention is in a sense the border case of a facet grinding with γ→0°.

Moreover, the cutting edge of the claimed cannula is approximately perpendicular to the plane of the basic or primary grinding, whereas the cutting edges of all previously known cannulas are in the plane of the primary grinding of the cannula. In case of the latter, the membrane or tissue cut by the cannula generally continues to rupture or tear open in the direction of the first cut and, thus, when inserting the grinding, the rear cutting edge inevitably strikes the edging of the pre-cut or ruptured opening, so that particles can be planed off by this edge (as described in more detail below). By contrast, the claimed cannula allows for a substantially perpendicular pre-cut in the membrane and, consequently, for an substantially perpendicular rupture when the grinding is inserted. As a result, an approximately triangular crotch or triangular portion is generated above the cannula grinding where the rear cutting edge of the grinding penetrates the material. This means that the rear cutting edge of the grinding does not strike an edging which could be planed off (see below).

The second advantage of the grinding according to the present invention is that the grinding lines of the beveled tip do not end in a pointed triangle with very small wall thicknesses (as is the case in the prior art), but in a cutting edge that extends over the cannula's entire wall thickness and which is therefore relatively stable. This is why in the case of port cannulas the above described, detrimental curled-up hook is not formed at the cannula tip. As a result, the disadvantageous punching effect can be avoided, while at the same time the disadvantageous hook formation and the subsequent damage of the silicone membrane by the hook can be prevented.

According to a preferred embodiment of the invention, the intersection line in which the second and third ground sections intersect, and the longitudinal cannula axis form an angle between 70° and 110°, preferably between 75° and 105°, more preferably between 80° and 100°, and particularly preferably between 85° and 95°. The above discussed advantages are most distinct when the angle included between the intersection line and the longitudinal cannula axis ranges between approximately 70° and approximately 110°. At an angle of around 90° puncturing forces can, in some cases, become too strong. Therefore, the angle formed by the intersection line and the longitudinal cannula axis preferably ranges between approximately 45° and approximately 80° or between approximately 100° and approximately 135°.

According to a further preferred embodiment, the second and third ground sections which are defined by the two section grindings form an angle between 20° and 90°, preferably between 25° and 75°, and particularly preferably between 30° and 60°. The first ground section and the cannula's longitudinal axis preferably form an angle between 8° and 20°, particularly preferably between 10° and 15°.

According to a particularly preferred embodiment, the cannula tip is bent inwardly in the direction of the cannula's longitudinal or center axis. This has the additional advantage that the one part of the grinding which is distant from the tip is virtually placed in the shadow of the cannula tip and, thus, no longer or only to a small extent cuts into the tissue or the membrane. In case of a bent cannula tip the above described angles between the intersection line and the longitudinal cannula axis, between the second and third ground sections and between the first ground section and the longitudinal cannula axis are to be understood such that these angles apply when the cannula is unbent. Naturally, the angles can deviate from those achieved during the grinding's manufacturing due to the bent configuration of the cannula tip. In particular, the basic grinding and the two section grindings do no longer define ground sections, but curved ground sections after bending of the cannula tip. From the final product with the bent cannula tip one can, however, still determine the original angles, i.e. before the bending process.

The cannula tip comprises a distal end. The cannula tip is preferably bent inwardly, i.e. in the direction of the cannula's longitudinal axis, such that the distal end of the cannula tip lies within the imaginary extension of the outer diameter of the unbent cannula. In other words, the bent cannula defines a cylinder which can be extended beyond the distal end of the cannula and comprises an inner and an outer diameter. The distal end of the cannula tip is to lie within the outer diameter of this cylinder after bending. At that, the distal end of the cannula tip preferably lies within the imaginary extension of the inner diameter of the unbent cannula, i.e. within the inner diameter of this cylinder. It is particularly preferred that the cannula tip is bent inwardly beyond the longitudinal cannula axis so that the distal end of the cannula tip lies between the cannula's longitudinal or center axis and the imaginary extension of the opposing inside cannula wall, i.e. between the center axis and the inner diameter of the (extended) cylinder.

It is furthermore preferred that the cannula tip is bent inwardly after grinding of the cannula. In this way, one, several or all ground sections preferably become curved ground sections as the surface(s) generated during the grinding process become(s) curved when bent inwardly. Hence, the final product preferably comprises one or more curved ground sections.

That way the above discussed angles can be generated in a defined manner, which simplifies the manufacturing process. As a result of the bending of the cannula tip the cutting edge is no longer perpendicular to the longitudinal cannula axis. However, it essentially maintains its shape when the cannula tip is bent, so that the cannula's cutting edge still enables perpendicular pre-cutting of the membrane and subsequent perpendicular rupture so that punching or planing of the membrane (or tissue) is effectively prevented. This applies when the cannula tip is not overly bent so that the cannula's distal end is located within the imaginary extension of the outer diameter of the unbent cannula, as described above.

The second advantage of the claimed grinding is also maintained irrespective of how far the cannula tip is bent inwardly, for also when the cutting edge is bent, it still extends over the entire wall thickness of the cannula and is correspondingly stable, and the formation of a hook at the cannula tip when hitting a port plate is prevented.

It is furthermore preferred that one or more sections of the grinding are blunted. It is particularly preferred that one or more sections which are distant to the tip, i.e. sections which are at least spaced 1 mm apart from the tip of the cannula grinding, are blunted or rounded.

The present invention further provides a method for manufacturing a cannula with a beveled end and, in particular, a cannula, as described above. Accordingly, the present invention provides a cannula with a longitudinal cannula axis provided with a basic grinding which defines a first ground section, and two section grindings which respectively define second and third ground sections. At that, the second and third ground sections intersect at one intersection line each, which is substantially perpendicular to the cannula's longitudinal axis. The three ground sections are preferably arranged such that the above discussed angle conditions are met.

The method preferably also comprises a further step in which the cannula tip is bent towards the longitudinal cannula axis, wherein the bending process follows after the grinding of the three ground sections. The bending is preferably carried out such that the above conditions regarding the claimed cannula are met.

Preferred embodiments of the invention are described in the following with respect to the drawings.

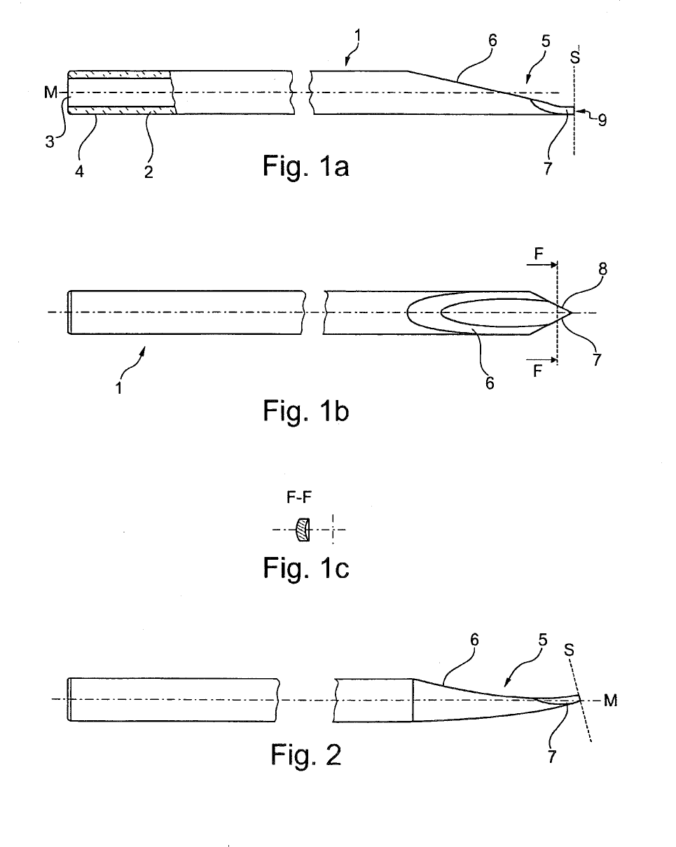

FIGS. 1a and 1b show a side view and a top view of a preferred embodiment of the cannula according to the present invention.

FIG. 1c shows an intersection through FIG. 1b along the line F-F.

FIG. 2 shows a side view of a further preferred embodiment of a cannula according to the present invention.

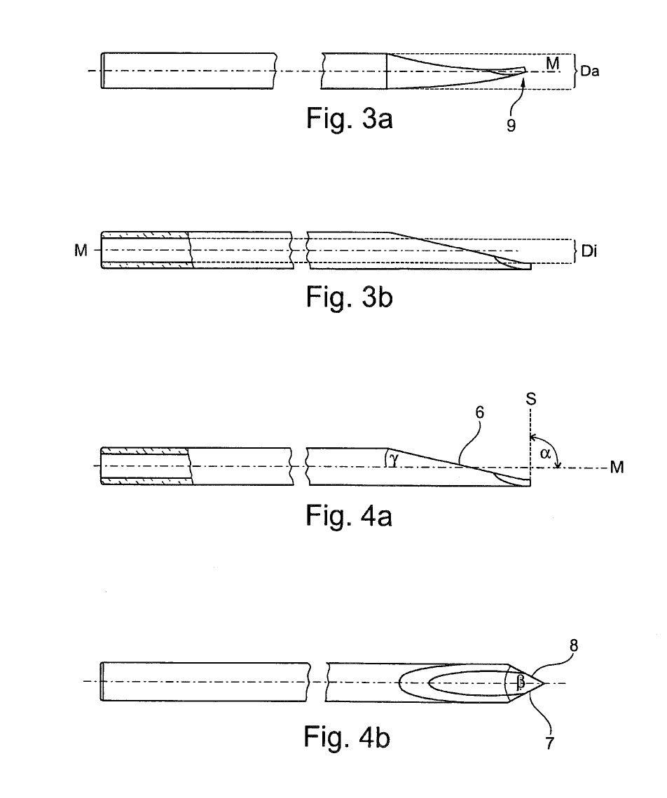

FIGS. 3a and 3b show side views of the embodiments of FIGS. 2 and 1.

FIGS. 4a and 4b show a side view and a top view of the embodiment according to FIG. 1.

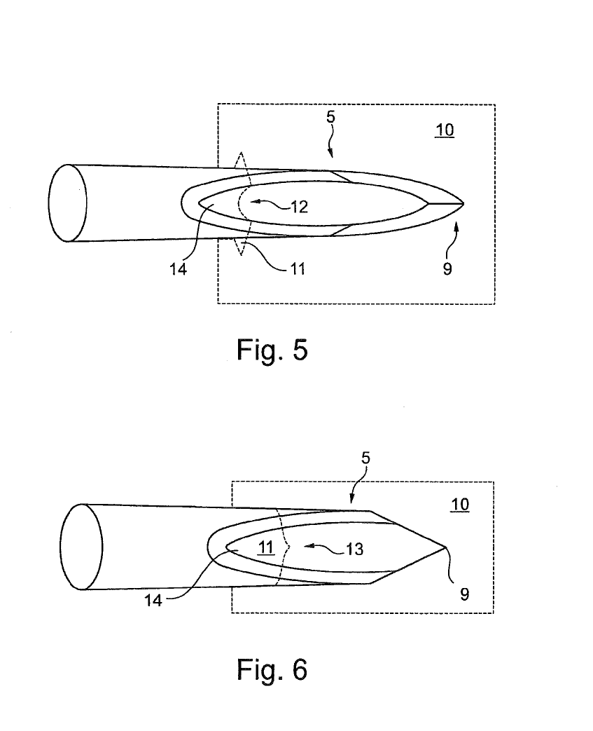

FIG. 5 schematically shows the cutting pattern generated by a prior art cannula.

FIG. 6 shows the cutting pattern generated by a cannula according to the present invention.

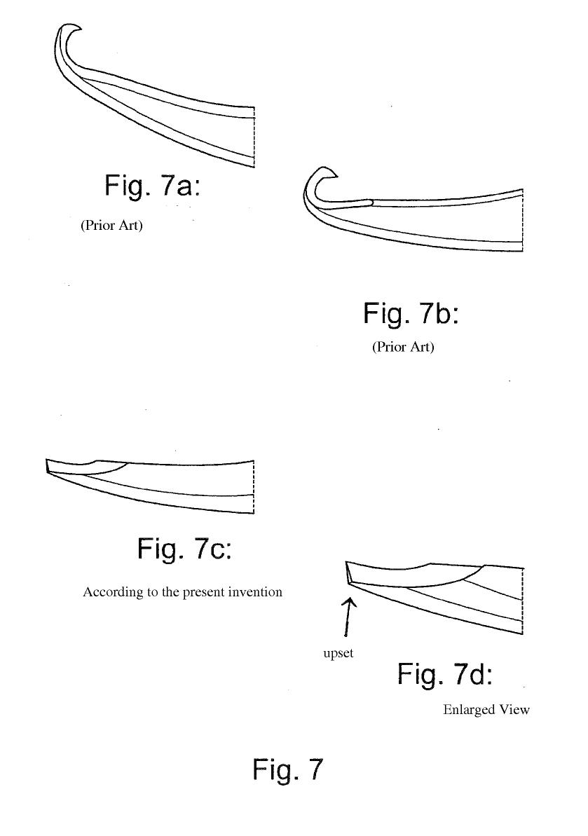

FIGS. 7a to 7d show photographs of different cannula tips after an injection performed with a force of 20 N.

FIGS. 1a and 4a show a respective side view of a preferred embodiment of the cannula according to the present invention. FIGS. 1b and 4b show the corresponding top view and FIG. 1c shows an intersection of FIG. 1b along the line F-F. The cannula 1 according to the present invention has a longitudinal or center axis M and a cannula tip 5. The cannula tip 5 comprises a beveled or ground end consisting of a basic or primary grinding 6 and two section grindings 7 and 8. The basic grinding 6 defines a first ground section (cf. FIGS. 1a and 4a) and the two section grindings 7 and 8 define a second and third ground section (cf. FIGS. 1b and 4b). The second and third ground sections intersect at an intersection line S which is substantially perpendicular to the longitudinal cannula axis M. In other words, the angle α which is formed by the intersection line S and the longitudinal axis M ranges between 45° and 135°.

The angle α preferably ranges between 70° and 110°, more preferably between 75° and 105°, even more preferably between 80° and 100°, and particularly preferably between 85° and 95°.

With the angle α being substantially perpendicular, the cannula grinding does not end in a triangular tip like with known cannulas, but in a sharp cutting edge 9 which is substantially perpendicular to the cannula wall 2 and whose length roughly corresponds to the wall thickness of the cannula. Moreover, the cutting edge 9 of the claimed cannula is approximately perpendicular to the surface 6 of the basic or primary grinding. The angle formed between the cutting edge and the surface of the primary grinding preferably ranges between 70° and 82°, and particularly preferably between 75° and 80°.

The second ground section 7 and the third ground section 8 form an angle β (cf. FIG. 4b) which preferably ranges between 20° and 90°, particularly preferably between 25° and 75° and most preferably between 30° and 60°. The first ground section 6 and the longitudinal cannula axis M preferably form an angle γ (cf. FIG. 4a) which preferably ranges between 8° and 20°, and particularly preferably between 10° and 15°.

It is preferred that one or more sections of the grinding are blunted. It is particularly preferred that one or more sections which are distant to the tip are blunted or rounded. Thus, for example, the section of the tip that is on the other side of or distal to the line F-F in FIG. 1b can be sharpened, while the section on this side of or proximal to the line F-F (or part of it) can be blunted or rounded.

FIGS. 2 and 3a show side views of a particularly preferred embodiment of the cannula according to the present invention. In this embodiment the cannula tip 5 of the claimed cannula 1 is bent inwardly towards the cannula's longitudinal axis M. In the concretely depicted case in FIG. 2 the cannula tip 5 is bent inwardly to such an extent that its distal end or the cutting edge 9 come to rest at the cannula's longitudinal or central axis M. The distal end or the cutting edge 9 are preferably located within the imaginary extension of the outer diameter Dₐ of the unbent cannula (cf. FIG. 3a). It is particularly preferred that the distal end or the cutting edge 9 lie within the imaginary extension of the inner diameter Dᵢ of the unbent cannula (cf. FIG. 3b for the definition of D₁). According to a preferred embodiment of the invention, the distal end of the cannula tip or its cutting edge 9 are located between the cannula's central axis M and the imaginary extension of the opposing inner wall 2. In other words, the cannula tip 5 is preferably bent to a greater extent than shown in FIGS. 2 and 3a.

As can be taken from FIG. 2, after bending of the cannula tip 5 the angle α between the intersection line S and the center axis M is no longer 90°, as depicted in FIG. 4a. Yet, in the embodiment describing a bent tip the indicated angles of the present invention are to be understood such that they describe the angles before bending, for these angles define the actual shape of the grinding and are crucial for the cutting properties.

Should angle α deviate from 90° already before bending, this deviation and the bending of the cannula tip can either have a compensating effect by setting upright a cutting edge which has a slightly forward inclination (angle α smaller than 90°) before bending, or an accumulative effect by bending and further tilting a cutting edge which has a slightly backward inclination (angle α greater than 90°).

Contrary to conventional cannula tips, the cannula tip according to the present invention comprises a significantly improved cutting pattern, which allows for a most efficient prevention of punching or planing of tissue or membrane material. In the following this shall be explained in more detail on the basis of the schematic illustrations of FIGS. 5 and 6.

The grey section 10 in FIGS. 5 and 6 is to indicate a silicone membrane which is, for instance, penetrated by a port cannula. The following remarks apply however analogously to the penetration of tissue. The puncturing of a silicone membrane (by a cannula) always starts with piercing the synthetic membrane (mostly made of silicone rubber) by means of the cutting edge of the cannula, said cut is then widened during penetration of the cannula grinding, which generally leads to further tearing in the direction of the first cut. All previously known cannulas have cutting edges which are located approximately in the plane of the cannulas' primary grinding. If one defines the one side of the cannula on which the tip of the grinding, i.e. the cutting edge, is located as the cannula's lower side, and the one side of the cannula on which the rear beveled end is located as the cannula's upper side (in other words, FIGS. 5 and 6 show a top view of the cannula's upper side), the first cut, in the case of conventional cannulas, will extend approximately perpendicular to the axis between the upper and the lower side. This leads to a horizontal first cut or pre-cut of the membrane by the grinding tip, as depicted in FIG. 5 by the incision 11 in the membrane 10. When the cannula is further inserted or advanced into the membrane after the first cut, a respective horizontal rupture will be the inevitable result. This way a membrane lug 12 is formed, which, when the cannula tip is further inserted into the membrane, will strike against the rear cutting edge, i.e. the rear beveled end which can then plane off membrane particles from the lug 12. Hence, cannula grindings known from the prior art lead to the above discussed punching or planing of membrane or tissue material.

By contrast, the cannula according to the present invention causes an substantially perpendicular first cut or pre-cut of the membrane by the cutting edge 9 (cf. FIG. 6) and, consequently, a perpendicular rupture when the cannula tip is further inserted into the membrane. Due to the perpendicular first cut an approximately triangular crotch 13 (cf. FIG. 6), instead of the lug 12 (cf. FIG. 5), is formed above the cannula grinding. This means that the rear cutting edge, i.e. the rear beveled end 14, of the cannula grinding does not strike against a protruding material lug 12 when the cannula is further inserted into the membrane, but slides below the crotch 13 without hitting membrane material that could be planed off. In this way the punching or planing of membrane or tissue material is most effectively prevented. As long as the vertical component of the intersection line between the second and third ground sections is greater than the horizontal component, the first cut or pre-cut predominantly extends in a perpendicular direction, which largely prevents punching or planing.

This positive effect can be further enhanced by bending the cannula tip towards the longitudinal cannula axis in accordance with the invention's preferred embodiment, for this shifts the crotch 13 further upwards relative to the beveled end 14 so that the beveled end 14 can even more securely slide below the crotch 13 without interacting with the membrane material.

A further advantage of the cannula according to the present invention is, as already explained above, that the grinding lines of the tip do not end in a pointed triangle with very small wall thicknesses (as is the case in the prior art), but in a cutting edge that extends over the cannula's entire wall thickness and which is therefore particularly stable. This is why the cannula according to the present invention does not form a curled-up hook at its tip when the cannula tip hits the ground plate of a port.

To illustrate this, two of Germany's best-selling punch-reducing cannulas and the cannula according to the present invention were pierced into an Intraport Keramik by Fresenius Kabi with an insertion force of 20 N. In order to clearly illustrate the effect, an insertion force was selected which is approximately twice the usual insertion force (about 10 N) applied on port cannulas.

FIGS. 7a to 7c show photographs of the cannula tips of the two known cannulas and of the claimed cannula after insertion. The two prior art port cannulas (cf. FIGS. 7a and 7b) form a very pronounced, curled-up hook at their tips. Obviously, such a hook can cause severe damage to the membrane or tissue when retracted. The cannula according to the present invention (cf. FIG. 7c), by comparison, forms no hook at all. In the enlargement of FIG. 7d a slight upset can be detected at best, although a force of 20 N was applied during the test, which is roughly twice the usual force occurring with port cannulas.

The above statements show that the new cannula grinding according to the present invention displays significant advantages over known cannulas comprising a facet grinding or rear bevel, because, on the one hand, the perpendicular cutting edge of the claimed cannula tip effectively prevents punching or planing of membrane or tissue material, while, on the other hand, the stable cutting edge of great wall thickness remains substantially dimensionally stable even under the application of great forces and does not tend to form hooks.

Although the advantageous stable configuration is of particular importance for the use as port cannula, the cannula according to the present invention is also perfectly suitable for other fields of application, in particular due to its superior capability of avoiding punching, which is always desired when sensitive material is punctured or when the cannula tip is inserted into a region where bacterial infections are critical. Hence, the cannula according to the present invention is, inter alia, also perfectly suitable for intraarticular puncturing or as spinal cannula.

1. A cannula having a longitudinal cannula axis and a cannula tip with a beveled end which comprises a basic grinding defining a first ground section and further comprises two section grindings, wherein the two section grindings respectively define second and third ground sections which intersect at an intersection line, wherein the intersection line and longitudinal cannula axis include an angle between 70° and 110°.

2. The cannula according to claim 1, wherein the intersection line and the longitudinal cannula axis include an angle between 75° and 105°, preferably between 80° and 100, and particularly preferably between 85° and 95°.

3. The cannula according to claim 1, wherein the second and third ground sections include an angle between 20° and 90°, preferably between 25° and 75°, and particularly preferably between 30° and 60°.

4. The cannula according to claim 1, wherein the first ground section and the longitudinal cannula axis include an angle between 8° and 20°, preferably between 10° and 15°.

5. The cannula according to claim 1, wherein the cannula tip is bent inwardly toward the longitudinal cannula axis.

6. The cannula according to claim 5, wherein a distal end of the cannula tip is located within the imaginary extension of the outer diameter of the unbent cannula.

7. The cannula according to claim 5, wherein a distal end of the cannula tip is located within the imaginary extension of the inner diameter of the unbent cannula.

8. The cannula according to claim 5, wherein a distal end of the cannula tip is located between the cannula's longitudinal cannula axis and the imaginary extension of the opposing inner wall.

9. The cannula according to claim 5, wherein the cannula tip was bent inwardly after providing the cannula with the beveled end, so that preferably one or more ground sections form curved ground sections.

10. The cannula according to claim 1, wherein one or more sections of the beveled end which are preferably distant to the tip are blunted.

11. A method for manufacturing a cannula with a beveled end, in particular a cannula according to any one of the preceding claims, comprising the steps of:
a) providing a cannula with a longitudinal cannula axis;
b) adding a basic grinding defining a first ground section; and
c) adding two ground sections which respectively define second and third ground sections;
wherein the second and third ground sections intersect at an intersection line which is essentially perpendicular to the longitudinal cannula axis and wherein the intersection line and the longitudinal cannula axis include an angle between 70° and 110°.

12. The method according to claim 11, wherein the intersection line and the longitudinal cannula axis include an angle between 75° and 105°, preferably between 80° and 100°, preferably between 80° and 100° and particularly preferably between 85° and 95°.

13. The method according to claim 11, further comprising step d) of bending the cannula tip toward the longitudinal cannula axis, wherein step d) is effected after steps b) and c).

14. The method according to claim 13, wherein the cannula tip is bent inwardly to such an extent that a distal end of the cannula tip is located within the imaginary extension of the outer diameter of the unbent cannula.

15. The method according to claim 13, wherein the cannula tip is bent inwardly to such an extent that a distal end of the cannula tip is located within the imaginary extension of the inner diameter of the unbent cannula.

16. The method according to claim 13, wherein a distal end of the cannula tip is located between the cannula's longitudinal cannula axis and the imaginary extension of the opposing inner wall.

17. The method according to claim 11, further comprising the step of:
blunting of one or more sections of the beveled end which are preferably distant to the tip.
